(19) United States
(12) Patent Application Publication
(10) Pub. No.: US 2026/0325650 A1
(43) Pub. Date: Oct. 22, 2026

(54) NATURAL EXCIPIENT HAVING MULTIFUNCTIONAL APPLICATION AND PROCESSES FOR THE PREPARATION THEREOF

(71) Applicant: REGERON, INC., Chuncheon-si (KR)

(72) Inventor: Kailas Kalicharan MORAVKAR, Chuncheon-si (KR)

(73) Assignee: REGERON, INC., Chuncheon-si (KR)

(21) Appl. No.: 19/721,718

(22) Filed: Jun. 26, 2026

Related U.S. Application Data
(63) Continuation of application No. 18/547,851, filed on Dec. 2, 2022.

(30) Foreign Application Priority Data
Dec. 2, 2021 (IN) 202121009068

(51) Int. Cl. A61K 47/46; A61K 8/9794; A61K 9/16; A61K 9/20; A61K 9/70; A61K 31/155; A61K 31/4178; A61K 47/34; A61K 47/38; A61K 47/44; A61Q 19/00

(52) U.S. Cl. CPC A61K 47/46; A61K 8/9794; A61K 9/1694; A61K 9/2018; A61K 9/2054; A61K 9/2068; A61K 9/7023; A61K 31/155; A61K 31/4178; A61K 47/34; A61K 47/38; A61K 47/44; A61Q 19/00; A61K 2800/10; A61K 2800/48

(57) ABSTRACT

A natural absorbent excipient having multifunction developed by a twin-screw hot melt extrusion process is disclosed. The present invention discloses a natural excipient composition comprising: (a) natural coconut husk and/or coir pith powder from the husk of coconut, the fruit of Cocos nucifera; and (b) at least one polyol compound consisting of glycerol, sorbitol, polyethene glycol (PEG), propylene glycol (PG), and combinations thereof, wherein the weight ratio of (a) to (b) is in the range of 1:1-6:4.

### CROSS-REFERENCE TO RELATED APPLICATIONS

This application is a continuation application of U.S. application Ser. No. 18/547,851, filed Aug. 24, 2023, which is a national stage of International Application No. PCT/KR2022/019441, filed Dec. 2, 2022, which claims the benefit of Indian Application No. 202121009068, filed Dec. 2, 2021, in the Indian Patent Office, the disclosures of which are incorporated herein by reference.

### TECHNICAL FIELD

The present invention relates to the continuous manufacturing of water-absorbent excipients using a twin-screw hot melt extrusion process. This invention also covers the continuous manufacturing of biodegradable natural water absorbents having multi-functional applications. The prepared functional excipients can be used in pharmaceuticals, nutraceuticals and cosmeceuticals. More specifically it pertains to a novel co-processed excipient obtained from the husk of the coconut, the fruit of Cocos nucifera to enhance the water- and/or any other fluid-absorbing property of different dosage forms.

### BACKGROUND ART

A therapeutic/non-therapeutic formulation consists of two parts-active principle (drug/active ingredient) and the inactive principle (excipient) responsible for binding, bulking up, masking bitterness etc. of the formulation. Binders, gums, sugars etc. are some common ‘excipients’ which are not active as therapeutic agents, but are required to be added in a dosage form, e.g., like a tablet, capsule, ointment, cream or even a syrup, etc.

Excipients provide a pivotal role in the development process of various dosage types and in their administration. An inappropriate option of an excipient can also lead to extreme intoxications, as witnessed by epileptic patients in Australia in the late 1960s who were taking phenytoin capsules (Furrer, P., 2013). The calcium sulphate used as a diluent in the capsule had been substituted by lactose and this replacement, perceived to be innocuous, resulted in safe delivery of phenytoin. Most formulations contain excipients at a higher concentration than the active principle and as a consequence, excipients contribute critically towards processing, stability, safety and performance of different formulations. The majority of the excipients that are currently available fail to meet the desired set of functionalities, therefore, creating urgency for the development of high functionality excipients. Broad functionality excipients could be accessed by inventing novel chemical excipients, better grades of approved excipients and new configurations of existing excipients. (Kaushik et al., 2012)

The chart below (FIG. 1) indicates the high functionality excipients present in the market. As indicated in the chart, some high functionality excipient simultaneously confers two or more functional attributes in their formulations, because, as in with Ludipress, the excipient can be used as a diluent as well as a lubricant, for example. Other co-processed excipients may also be widely applicable. Colloidal silicon dioxide has been used nearly 70 years as an absorbent. The first scientific literature on the use of AEROSIL® fumed silica as a pharmaceutical excipient was published in 1957. Since then, ‘AEROSIL® 200 Pharma’ has gained acceptance and recognition as a pharmaceutical excipient. People have seen serious side effects with silicon dioxide when used as an absorbent in different formulations, though the U.S. Food and Drug Administration (FDA) had already identified silicon dioxide as a safe food additive. In 2018, the European Food Safety Authority recommended implementation of stricter guidelines on silicon dioxide uses to the European Union before more studies could be performed. (EFSA journal)

The coconut husk and/or coir pith powder is one of the natural lignocellulosic fibres that are commonly used in many applications today. A tropical plant of the Arecaceae (Palmae) family, coconut, the fruit of Cocos nucifera, is extensively grown in coastal regions of tropical countries. In Malaysia, coconut seems to be the fourth most traditionally producing crop after palm oil, rubber and paddy. Coconut husk is present in substantial amounts as residues from the processing of coconut in many regions, resulting in various coarse coir fibres, which are seed-hair fibres derived from the outer coconut shell. As there is plenty of coconut husk and/or coir pith powder available, potentially valuable lignocellulosic raw materials for conversion into pulp and paper could be seen from sources of the supply of the raw materials. (Main et al., 2014)

Though many potential benefits of the coconut husk and/or coir pith powder could be exploited, this raw material has not been fully utilised for productive purposes in spite of its availability in significant quantities. Substantial amounts of coconut husk and/or coir pith powder accumulate nearby coir processing units per year, creating serious disposal problems and fire risks. Owing to its high lignin and cellulose content (approximately 40%) and high polyphenol content (approximately 100 mg/100 g coir pith), its degradation and mineralization rates are very slow under natural conditions, imparting longer stability for its compounds. India has the capacity to produce about 280,000 metric tons of coconut husk and/or coir pith powder. It can become the key source for indigenous use and exports of this valuable organic resource. Lignin prevents easy decomposition and mineralization of coconut husk and/or coir pith powder. Lignin is a three-dimensional amorphous aromatic heteropolymer comprising stable C—C, ether-ester linkages between monomeric single- or double-methoxylated phenyl propyl phenolic units that render it a biopolymer recalcitrant. Degradation of lignin often relies on the availability of a co-substrate such as glucose, which is readily used to metabolize it. (Prabhu et al., 2002)

Lignin is a class of complex organic polymers that form key structural materials, and, in the construction of cell walls, lignin is especially important in wood and bark in particular, since it provides rigidity and does not rot easily. Chemically speaking, lignins are cross-linked phenolic polymers. Lignin is the second most abundant biopolymer of lignocellulosic biomass, after cellulose. Lignin is tightly bound by non-covalent forces or covalent bonds to form complexes of carbohydrates with cellulose and hemicellulose. Lignin has diverse pharmacological activities, such as anti-tumour, anti-hyperglycemic, antimicrobial, anti-HIV and antioxidant activities; however, in contrast to polysaccharide-based materials, lignin has not yet been exploited significantly in the biomedical field. (Spiridon et al., 2018)

The name coconut (Cocos nucifera) is derived from Portuguese word coco, meaning “head” or “skull” and nucifera is derived from the Latin words nux (nut) andfera (bearing), for ‘nut-bearing’. Cocos nucifera belongs to the Arecaceae (palm) plant family. There are 2 types of coir fibres, namely, brown fibres and white fibres. Brown fibres are derived from full-blown coconuts with properties such as thickness, durability, and high abrasion resistance, and are most commonly used in engineering work. Whereas, in addition to poorer strength, white fibres derived from adolescent coconuts are smoother and finer. It is noted that by incorporating fibres into the concrete, the concrete strength could be marginally increased due to coconut coir's high lignin and low cellulose nature, rendering it solidity as well as producing extra interfacial transition area within the concrete that may have an effect on compressive strength. By adding coir fibres to the optimal concrete forming stage, it could be shown that an improvement in tensile strength was achieved. (Dhanasree et al., 2019)

Coconut husk and/or coir pith powder is a spongy, fluffy, light substance with improved and highly compressive water-holding capacity. The pH is similar to neutral for the composted coir pith, while the pH of the normal pith is acidic. (Ghosh et al., 2007)

Co-processing here refers to the use of waste as a renewable raw material or an energy source, or both (material recycling), together with or without some other raw materials. Co-processing is the integration of two or more raw materials (comprising waste) that can shape the materials of superior functionality with optimal constituents ratios resulting in minimal unnecessary residual materials. Co-processing is essentially based on particle engineering that enables the primary excipients to change their Critical Material Attributes (CMA). These improvements should be mirrored as improved features in the resulting co-processed content.

### DISCLOSURE

### Technical Problem

The present invention discloses one such novel excipient obtained from the processing of natural sources which by itself is not an ‘active principle’ but which, when added to formulations, can enhance their efficacy by various aspects.

It has been already perceived that industrially scalable, continuous manufacturing of novel excipients obtained from the co-processing of natural sources and their combinations has never been disclosed earlier.

The principal objective is to develop a novel biodegradable natural excipient that functions as an absorbent as well as a lubricant in the design of a variety of pharmaceutical, nutraceuticals and/or cosmaceuticals formulations, which could be a good alternative to synthetic silicon-based excipients.

Another object of the proposed work relates to the development of biodegradable natural excipients by twin-screw hot melt extrusion technology as a one-step, non-aqueous, industry-scalable and continuous manufacturing process, so as to avoid the need for large amounts of water to be evaporated during wet conditions like wet granulation, saving production times and costs, especially in the formulations sensitive to humidity and heat.

It is another object of the present invention to provide an excipient with good water absorption and lubricant properties that retains moisture and prevents drying-up resulting in ease of handling, packaging, logistics, and prolonged shelf life. The present biodegradable natural water-absorbent (i.e., coconut husk and/or coir pith powder from the husk of coconut) has a water-absorbing power of at least 8-10 times its own weight within 3 h of exposure to water.

An additional object of the present invention is to provide a natural excipient with multiple applications like thickener, softener, rheology controller and storage stability enhancer in semi-solid and liquid dosage forms and as a source of dietary fibre in the design of a variety of pharmaceutical, nutraceutical and/or cosmeceutical formulations.

Another object of the present invention is to disclose that the novel excipient is biodegradable natural coconut husk and/or coir pith powder from the husk of coconut, the fruit of Cocos nucifera which is co-processed with at least one polyol compound. This co-processed excipient obtained from a biodegradable natural source, that is, the husk of coconut (the fruit of Cocos nucifera), may replace synthetic (and/or natural) absorbents or lubricants used in the production of different solid, semi-solid and/or liquid formulations by way of its co-processing with at least one polyol compound, as the same excipient material (i.e., coconut husk and/or coir pith powder) can function as both absorbent and lubricant in the formulation. Therefore, this material is ideal for the preparation of different solid, semi-solid and/or liquid formulations requiring both water and/or any other fluid-absorbing as well as lubricating properties simultaneously.

Another object of the present invention is to develop biocompatible transdermal patches involving the use of proposed natural excipient to high load, to hold and release the active material in a controlled manner to elicit desirable therapeutic, nutraceutical and cosmetic action, particularly to supply medication locally to relieve the pain due to Rheumatoid arthritis (RA) using known anti-inflammatory oils and other active pharmaceutical ingredients (API's).

Another object of the present invention is to develop a natural excipient-based solid self-microemulsifying drug delivery system (SMEDDS) powder for efficient treatment of topical antifungal infection, which could have high percent loading and solubility of actives.

Another object of the present invention is to develop a novel biocompatible self-adhesive hydrogel film platform using natural excipients which could overcome the process limitations of current formulations on wound healing and skin rejuvenation/anti-ageing properties in cosmeceuticals.

### Technical Solution

A biodegradable natural absorbent excipient having multifunction developed by a twin-screw hot melt extrusion process is disclosed here.

In an aspect of the present disclosure, there is provided an natural excipient composition comprising: (a) natural coconut husk and/or coir pith powder from the husk of coconut, the fruit of Cocos nucifera; and (b) at least one polyol compound consisting of glycerol, sorbitol, polyethylene glycol (PEG), propylene glycol (PG), and combinations thereof, wherein the weight ratio of (a) to (b) is in the range of 1:1-6:4.

In an aspect of the present disclosure, there is provided a process for the preparation of a natural excipient composition (FIG. 2) comprising: (a) natural coconut husk and/or coir pith powder from the husk of coconut, the fruit of Cocos nucifera; and (b) at least one polyol compound consisting of glycerol, sorbitol, polyethene glycol (PEG), propylene glycol (PG), and combinations thereof, wherein the weight ratio of (a) to (b) is in the range of 1:1-6:4, and said process comprising the steps of: (c) The rotary cutter mill with standard sieve is used to make coir pith and/or their powder from the husk of coconut, the fruit of Cocos nucifera by cutting and size reduction process; and (d) Mixing of coconut husk and/or coir pith powder and at least one polyol compound consisting of glycerol, sorbitol, polyethene glycol (PEG), propylene glycol (PG), and combinations thereof in a gravimetric hopper; and (e) processing the mixture in the hot condition through twin-screw hot melt extrusion, wherein, a uniform product can be obtained with desired attributes by optimization of process parameters, and the ratio of the mixture.

### DESCRIPTION OF DRAWINGS

FIG. 1: Depicts some high functionality excipients present in the market.

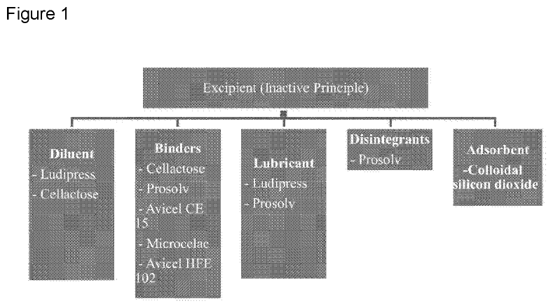

FIG. 2: Experimental design to prepare the co-processed multifunctional novel excipient.

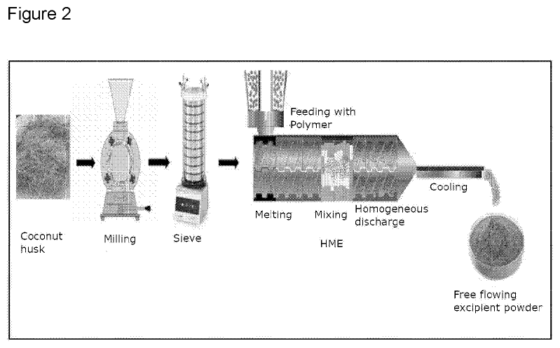

FIG. 3: Depicts the FTIR analysis of the functional excipient described in the present invention.

FIG. 4: SEM of the co-processed multifunctional novel excipient

FIG. 5: Particle size distribution of the co-processed multifunctional novel excipient

FIG. 6: In-vitro drug release study of formulation batch F6 by HPTLC batch densitograms

FIG. 7: Paw edema measurements: a) 0th minute; b) 60th minute; c) 120th minute; d) 180th minute; e) 240th minute (Note: Data expressed as mean±sem and analysed by One-way analysis of Variance followed by Bonferroni's multiple comparison post-test).

FIG. 8: IL-6 measurements of the transdermal patches (Note: Data expressed as mean±sem and analysed by One-way analysis of Variance followed by Bonferroni's multiple comparison post-test).

FIG. 9: PXRD patterns of the formulations and the raw material.

FIG. 10: Anti-fungal activity by petri-plate method.

FIG. 11: Skin irritancy test results: A) initial; B) after 24 h; C) after 48 h; D) after 72 h.

### BEST MODE

### Definitions

For convenience, before further description of the present disclosure, certain terms employed in the specification, and examples are collected here. These definitions should be read in the light of the remainder of the disclosure and understood as by a person of skill in the art. The terms used herein have the meanings recognized and known to those of skill in the art, however, for convenience and completeness, particular terms and their meanings are set forth below

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

The term “at least one” is used to mean one or more and thus includes individual components as well as mixtures/combinations.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

“Hot melt extrusion” (HME) is the process of applying heat and pressure to prepare co-processed excipient and force it through an orifice in a continuous process to obtain the product. HME is carried out using an extruder—a barrel containing one (single screw) or two co/counter-rotating screws (twin-screw) that transport material down the barrel.

The term “co-processing” or “co-processed” refers to the use of waste as a renewable raw material or an energy source, or both (material recycling), together with or without some other raw materials. Co-processing is the integration of two or more raw materials (comprising waste) that can shape the materials of superior functionality with optimal constituent's ratios resulting in minimal unnecessary residual materials.

The present disclosure is not to be limited in scope by the specific implementations described herein, which are intended for the purposes of exemplification only. Functionally equivalent products, compositions, and methods are clearly within the scope of the disclosure, as described herein.

Hot melt extrusion (HME) is an emerging, solvent-less polymer processing approach that both industry and academia are currently exploring as a means of delivering assorted dosage forms with enhanced final product compatibility. Furthermore, HME, by reducing production steps, provides the ability for faster and more productive cycles for the finished product.

Functional excipients have been formulated by hot-melt extrusion process to modify/superiority into their basic properties. (Crowley et al., 2007, Li et al., 2015)

In an embodiment of the present disclosure, natural coconut husk and/or coir pith powder from the husk of coconut, the fruit of Cocos nucifera and at least one polyol compound consisting of glycerol, sorbitol, polyethylene glycol (PEG), propylene glycol (PG), and combinations thereof were used to prepare a multifunctional novel excipient using twin-screw hot melt extrusion.

In an embodiment of the present disclosure, natural coconut husk and/or coir pith powder from the husk of coconut, the fruit of Cocos nucifera and at least one polyol compound consisting of glycerol, sorbitol, polyethylene glycol (PEG), propylene glycol (PG), and combinations thereof were used to prepare a multifunctional novel excipient using twin-screw hot melt extrusion wherein the temperature range of the hot melt extrusion is 60-110° C and screw speed were in the range of 25-60 rpm.

The details of the optimization ratio are given as the following Table 1:

TABLE 1
Optimization ration of natural coconut husk and/or coir pith
powder from the husk of coconut, the fruit of Cocos nucifera,
and at least one polyol compound

| | | | |
|---|---|---|---|
| | | Carr's | Water absorbing/ |
| | Physical | Index | holding capacity |
| Ratio | characteristics | (%) | (after 3 h) |

Coconut husk and/or coir pith powder: PEG

| | | | |
|---|---|---|---|
| 10:0 | Charred | — | — |
| 9:1 | | | |
| 8:2 | | | |
| 7:3 | | | |
| 6:4 to 1:1 | No change in color | 28 | 7.8 times of |
| | | | its own weight |

Coconut husk and/or coir pith powder: PG

| | | | |
|---|---|---|---|
| 9:1 | Charred | — | — |
| 8:2 | | | |
| 7:3 | No change in color | 23 | Between 6-8 times |
| 6:4 to 1:1 | | 19 | of its own weight |

Coconut husk and/or coir pith powder: Sorbitol

| | | | |
|---|---|---|---|
| 9:1 | Charred | — | — |
| 8:2 | | | |
| 7:3 | No change in color | 21 | Between 6-8 times |
| 6:4 to 1:1 | | 15 | of its own weight |

Coconut husk and/or coir pith powder: Glycerol

| | | | |
|---|---|---|---|
| 9:1 | Charred | — | — |
| 8:2 | No change in color | 18 | Between 6-10 times |
| 7:3 | | 12 | of its own weight |
| 6:4 to 1:1 | | 13 | |

In an embodiment of the present disclosure, based on the observations made in the Table 1, natural coconut husk and/or coir pith powder from the husk of coconut, the fruit of Cocos nucifera and at least one polyol compound consisting of glycerol, sorbitol, polyethylene glycol (PEG), propylene glycol (PG), and combinations thereof were selected in the range of ration 1:1 to 6:4 respectively to prepare multifunctional novel excipient using twin-screw hot melt extrusion.

In an embodiment of the present disclosure, more specifically, natural coconut husk and/or coir pith powder from the husk of coconut, the fruit of Cocos nucifera and at least one polyol compound consisting of glycerol and combinations thereof, were used in the range of 1:1 to 6:4 as a final optimized ration for further characterization and formulation.

In an embodiment of the present disclosure, a simple polyol compound is a glycerol (also called glycerine or glycerin). It is a colourless, odourless, viscous liquid that is non-toxic and sweet-tasting. In such lipids referred to as glycerides, the glycerol backbone is found. It is widely used in FDA-approved wound and burn treatments because it has antimicrobial and antiviral properties. It can also be used to measure liver disease as an effective marker. It is still commonly used in the food industry as a sweetener and in prescription formulations as a moisturiser. Glycerol is mixable with water due to the inclusion of three hydroxyl groups and is hygroscopic in nature. (en.wikipedia.org).

In an embodiment of the present disclosure, natural coconut husk and/or coir pith powder from the husk of coconut, the fruit of Cocos nucifera and at least one polyol compound consisting of glycerol, sorbitol, polyethylene glycol (PEG), propylene glycol (PG), and combinations thereof were selected in the range of ration 1:1 to 6:4, respectively to prepare multifunctional novel excipient using twin-screw hot melt extrusion, wherein the Carr's index of the said ration was found in the range of 28 to 13%.

In an embodiment of the present disclosure, natural coconut husk and/or coir pith powder from the husk of coconut, the fruit of Cocos nucifera and at least one polyol compound consisting of glycerol, sorbitol, polyethylene glycol (PEG), propylene glycol (PG), and combinations thereof were selected in the range of ration 1:1 to 6:4, respectively to prepare multifunctional novel excipient using twin-screw hot melt extrusion, the Carr's index was found in the range of 28 to 13%, wherein the water holding capacity of the said ration was found in the range of 6 to 10 times of its own weight.

In an embodiment of the present disclosure, the prepared co-processed multifunctional novel excipient using twin-screw extrusion was evaluated for parameters like physical characteristics, FTIR study, water absorbing/holding capacity and toxicity. The details of the evaluations are as follows:

### a) Physical Characteristics:

Physical characteristics of the prepared co-processed multifunctional novel excipient using twin-screw extrusion are given in the Table 2.

TABLE 2
Physical characteristics of co-processed
multifunction novel excipient

| | | |
|---|---|---|
| Bulk density | 0.04-0.05 | g/cc |
| Particle density | 0.8-1.2 | g/cc |

| | |
|---|---|
| Porosity | 95 ± 2.1% |
| pH | 6.8-7.2 |

### b) FT-IR Analysis of the Samples:

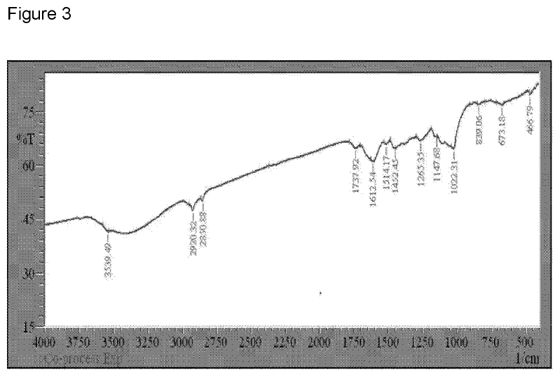

The infrared (IR) spectrum of organic molecules yields valuable information on the different functional groups present in them. In the present study, the IR spectra of natural coconut husk and/or coir pith powder from the husk of coconut, the fruit of Cocos nucifera and at least one polyol compound were recorded using a diffuse reflectance assembly. Prior to that, the sample was ground to a fine powder with dry KBr and the homogeneous solid solution containing 1-2% of the substance was used for recording IR spectra. FIG. 3 shows the IR spectra of the treated functional excipient sample. The major peaks are 3400, 2900, 1730, 1600 & 1510 cm⁻¹. The peaks in the region 2800-3000 and 3300-4000 cm⁻¹ arise from aliphatic C—H and O—H stretching vibrations. The values at 1510 and 1600 cm⁻¹ could be aromatic skeletal vibrations (of lignin), while the small absorption at 1730 cm⁻¹ may be due to ester groups (minor component).

### c) Water Holding Capacity

Water holding capacity is defined by the volume of water bound to the fibres comprising the usable excipient without any external force being applied (except for gravity and atmospheric pressure). Accurately weighed dry samples (1 g) were taken in a two graduated test tubes, about 30 ml of water was applied and hydrated for 3 h and 18 h respectively. The supernatant was extracted by going through the vacuum of the sintered glass crucible (G4). The weight of the hydrated residue was documented and dried for 2 h at 105° C to achieve residual dry weight. Water holding capacity (g/g)=(Residue hydrated weight−Residue dry weight)/Residue dry weight. (Thibault, J. F. et al) The water holding capacity was found approx. the range between, 6-10 and 15-20 times respectively to its own weight of functional excipient.

### d) Toxicity:

Toxicity studies in rats showed no toxic effects till the 72 h study. As this functional excipient had not been reported in the literature anywhere as an excipient, its toxicity studies were done as a priority to support the use of this novel functional excipient in pharmaceuticals, nutraceuticals and/or cosmeceuticals.

### e) Scanning Electron Microscopy (SEM)

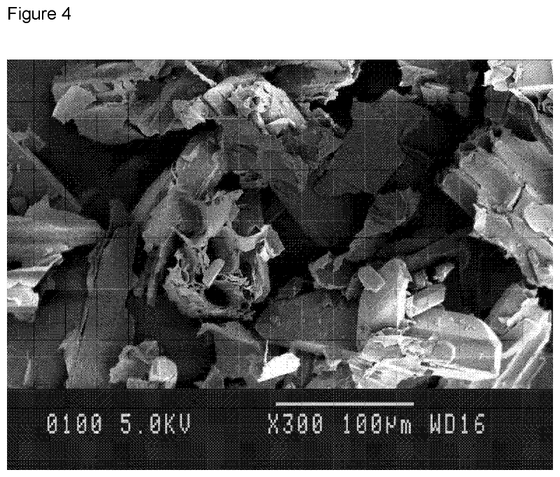

In order to observe the surface characteristics like porosity, particle size, etc; of co-processed multifunctional novel excipient prepared using twin-screw hot melt extrusion, surface images were captured using SEM. As shown in the FIG. 4, smooth surface and high porosity were observed in the SEM image at 10 kV, 300× magnifications.

### f) Particle Size Distribution

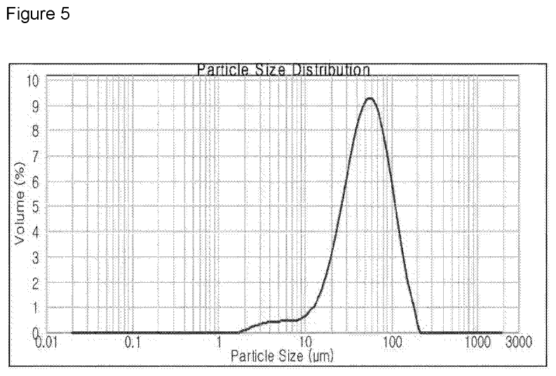

The Mean particle size analysis of the co-processed multifunctional novel excipient prepared using twin-screw hot melt extrusion technology was performed by using a Mastersizer 2000 MU (Malvern Instruments, UK). Sample preparation was done as per the suitable standard operating procedure (SOP) (approx. 0.3-0.5 g sample dispersed in 900 ml of suitable solvent (water and ethanol with the aid of sonication). The particle size distribution was observed in terms of d10—19.338 μm, d50—51.30 μm, and d90—110.49 μm as shown in FIG. 5.

From all the above-depicted results, it can be concluded that the co-processed multifunctional novel excipient prepared using twin-screw extrusion show excellent water absorbing/holding capacity due to high porosity and self-lubricating property, as evidenced by porosity and Carr's index respectively.

In an embodiment of the present disclosure, the example described below illustrates various aspects of the co-processed multifunctional novel excipient prepared using twin-screw extrusion and is not limited only to the claims.

### EXAMPLE

### Example 1: Drying Agent/Desiccator During Wet Granulation of Metformin HCl IR Tablets

The co-processed multifunctional novel excipient described above is an efficient desiccant. For example, it can be used in wet granulation processes thus avoiding the need for large amounts of water to be evaporated. The drug was mixed individually with lactose, and croscarmellose sodium. A solution of polymeric binder in water was prepared to be used as a binder. A wet mass of the drug-excipient mixture was prepared using the binder solution. The wet mass was sieved using suitable sieves of mesh sizes #6-12 screens to obtain granules. The moist granules were dried at 40-50° C for 30 min and screened through sieves of #14 to 20 mesh size. The dried sieved granules were mixed with lubricant. Both drying and milling steps in wet granulation were not required for the batch I formulation. Carr's index, angle of repose, and Hausner's ratio of granules were found in the range of excellent to good flowability as per USP limits.

TABLE 3
Formulae Metformin HCl IR tablet

| Ingredients | Batch I (% w/w) | Batch II (% w/w) |
|---|---|---|
| Metformin HCl | 70 | 70 |
| Hydroxypropyl cellulose | 10 | 10 |
| Lactose | 10 | 10 |
| Croscarmallose sodium | 6.5 | 6.5 |
| Co-processed multifunctional | 1.5 | — |
| excipient | | |
| Magnesium stearate | — | 1.5 |
| Water | 2 | 2 |
| Total | 100 | 100 |

### Example 2: Thickening Agent for Topical Application to an External Portion of Skin (Human/Animal)

In an embodiment of the present disclosure, there is provided functional excipient used as thickening agent for the preparation of semi-solid dosage forms like ointment, gel, cream, etc;. It can be used as a pharmaceutical, neutraceutical, and/or cosmeceutical topical application to an external portion of skin (human/animal). Preparation of such a thickening agent includes ingredients consisting of:
a. 0.5 to 3%, preferably 0.8 to 1.5% by weight of various coconut husk and/or coir pith powder;
b. 97 to 99.5%, preferably 98 to 99.2% by weight of water/any suitable fluid as vehicle.
   Table 4 comprises the results of prepared thickening agent.

TABLE 4
Property evaluation of thickening agent

| Formulation | pH | Spreadability | Texture profile |
|---|---|---|---|
| Thickening agent | 7.4 | Spreadable | Smooth |

### Example 3: Design and Development of Transdermal Patches to Relieve Rheumatoid Arthritis (RA)

The transdermal patch containing ginseng oil (8.4±0.12 ml/g of absorbent) on an absorbent was prepared by using HPMC and PEG-400 by the solvent casting method. Polymer was dissolved in 20 ml hydroalcoholic solution and placed on the magnetic stirrer with 60 rpm. Co-processed multifunctional novel excipient loaded with ginseng oil was transferred to the above mixture in the presence of PEG-400 (plasticizer) and DMSO (penetration enhancer). Mixture was poured in a glass Petri dish of 36.29 cm² area after volume make-up of 25 ml and subjected for drying at room temperature for 24 h. Patch was cut into the sizes in such a way that each patch contains the 1.2 ml of oil (2×2 cm²). Prepared patches were stored in a desiccator for further evaluation. To finalize the concentration of HPMC and PEG, six patches were prepared with different concentrations of HPMC and PEG (Table 5).

TABLE 5
Composition of the prepared patches

| | Formulation batches | | | | | |
|---|---|---|---|---|---|---|
| Ingredients | F1 | F2 | F3 | F4 | F5 | F6 |
| Co-processed excipient (mg) | 200 | 200 | 200 | 200 | 200 | 200 |
| Hydroxypropyl methyl cellulose (HPMC) (mg) | 320 | 250 | 300 | 179 | 300 | 250 |
| PEG-400 (mL) (30% w/w of polymers) | 2 | 2.71 | 2.50 | 1.50 | 2.00 | 1.50 |
| DMSO | 0.2 | 0.2 | 0.2 | 0.2 | 0.2 | 0.2 |

Prepared patches were evaluated for various parameters in triplicate and results as mean±SD was calculated. Thickness of prepared formulation was checked by using Digimatic Micrometer (Mitutoyo, ABSOLUTE) at different position of the patch in triplicate. It was found in the range 0.32±0.0447 mm to 0.34±0.0537 mm. No impact of concentration of HPMC as well PEG was observed on thickness of the patch. Prepared formulation was subjected to a weight variation test. Three patches from all batches were selected randomly and weighed. Average weight was found to be in the range of 0.193±0.032578 to 0.220±0.04875 gm/2 sq. inch (2×2). It was found that, as the concentration of polymer increased, so did the weight of patch. Prepared patches were evaluated by folding endurance test by continually folding the patch at the same location (NMT 300 folds) until it broke. The folding endurance of the patch was found to be in the range of 145±0.20 to 158±0.25. Change in concentration of HPMC as well as PEG showed impact on folding endurance. Tensile strength of the patch was determined with “CT-3 Texture analyzer” testing machine. Tensile strength was found to be in range 2.425±0.15 to 3.370±0.16 (MPa/mm). Concentration of polymer as well as plasticizer showed positive impact on the tensile strength. Prepared formulation was then investigated for percent moisture loss by placing the prepared patch in a desiccator in the presence of anhydrous calcium chloride. After 72 h, the films were checked for change in the weight. Percent moisture loss was calculated by using equation (1). Moisture contents for all developed formulations were found to be in the range of 2.252±0.90 to 7.151±1.44% as shown in the Table 6. Moisture contents in the patches were found to be in the optimum levels which maintain the stability as well as prevent them from complete dryness and breakdown. All the results are depicted in the Table 5.

$$Percentage Moisture loss = \frac{Initial weight - Final weigh}{Initial weight} \times 100 \qquad ( 1 )$$

TABLE 6
Evaluation of prepared formulation batches (n = 3, results: mean ± SD)

| Formulation | Average thickness | Average weight | Folding endurance | Tensile strength | Moisture content |
|---|---|---|---|---|---|
| batches | (mm) | (gm/2 sq. inch) | (Average number) | (MPA/mm) | % |
| F1 | 0.30 ± 0.16 | 0.188 ± 0.46 | 154 ± 0.19 | 2.425 ± 0.15 | 4.777 ± 1.05 |
| F2 | 0.37 ± 0.08 | 0.195 ± 0.97 | 158 ± 0.25 | 2.721 ± 0.18 | 4.097 ± 0.70 |
| F3 | 0.34 ± 0.04 | 0.190 ± 0.32 | 149 ± 0.17 | 2.917 ± 0.20 | 5.663 ± 1.39 |
| F4 | 0.33 ± 0.12 | 0.192 ± 0.46 | 147 ± 0.22 | 3.012 ± 0.23 | 7.151 ± 1.44 |
| F5 | 0.38 ± 0.10 | 0.215 ± 0.41 | 145 ± 0.20 | 3.370 ± 0.16 | 3.770 ± 0.81 |
| F6 | 0.36 ± 0.17 | 0.233 ± 0.30 | 152 ± 0.18 | 2.817 ± 0.19 | 2.252 ± 0.90 |

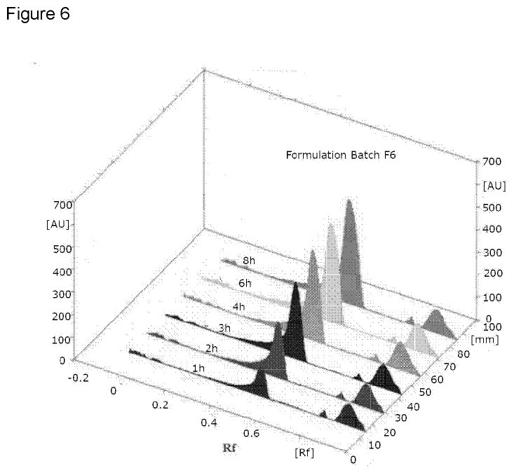

Further, the oil release from patch was confirmed prior to preclinical trials by using Franz diffusion cell. Alcoholic phosphate buffer (pH 7.4) was used as the diffusion medium. Prepared patch was placed on cellophane membrane (previously soaked in buffer for 8 h) and carefully clamped between donor and receptor compartments. Whole assembly was placed on magnetic stirrer and maintained at 32±0.5 °C. The samples (1 ml each) were withdrawn at 1, 2, 3, 4, 6, 8, 10 and 12 hours and analyzed by HPTLC; densitometry scanning was performed on Cagmag TLC scanner 3 in absorbance mode, and operated by winCATS software at 374 nm. The sink condition was maintained by adding the equal volume of buffer in receptor compartment. In vitro oil release study reveals that there is increase in ginseng oil release from prepared patch at certain interval of time up to 8 h (6.18±0.12%, 19.56±1.52%, 32.14±1.87%, 45.31±2.04%, 76.28±2.11%, and 99.41±1.31% respectively) and then release diminished, i.e., total release obtained (FIG. 6). Based on obtained results, batch F6 was considered as the optimized batch and it was subjected to preclinical studies.

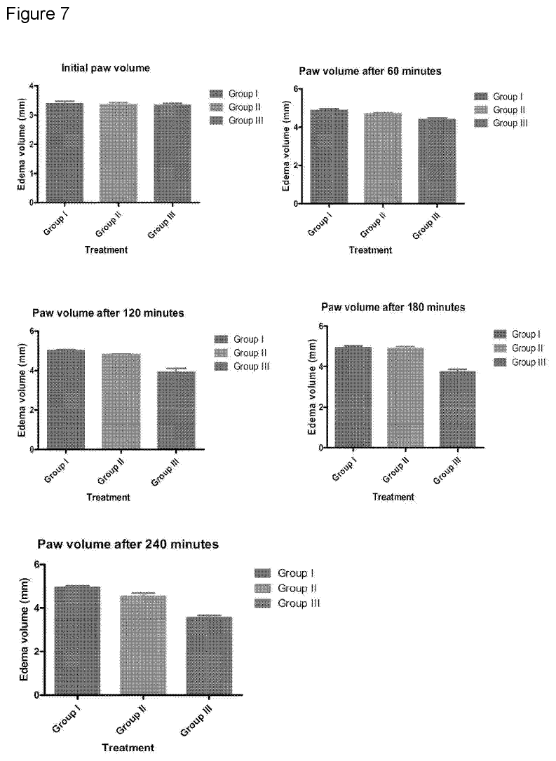

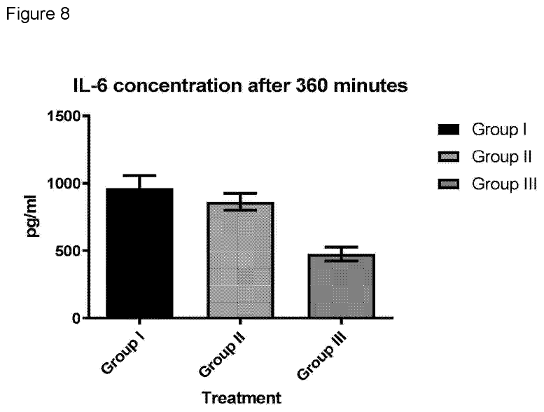

To check the effectiveness of an optimized ginseng oil-loaded transdermal patch, the anti-inflammatory test (carrageenan induced hind paw edema method) was carried out on 18 male Wistar rats of weight 142 to 151 g. The animals were kept under standard laboratory conditions and diet as per guidelines. Animals were divided in three groups randomly as control group (I), oil treated group (II) and patch treated group (III). Edema was induced by 0.1 ml of 1% λ-carrageenan-saline solution injection (22049 SIGMA λ-Carrageenan plant-mucopolysaccharide, Sigma-Aldrich). The thickness of paw edema was measured by using a standard Digital Vernier caliper for 4 h. Formulation showed better reduction in paw edema in comparison to application of pure oil. Results are shown in FIG. 7. After 6 h, animals were anesthetized and sacrificed by cervical dislocation. Blood samples were collected from treatment sites and analyzed for IL-6 by using ELISA kit as per instructions by the manufacturer. We found marked increase in the level of IL-6 in group I and II animals in comparison to group III animals (FIG. 8). Increase in IL-6 is the early key marker of arthritis. Obtained results further support the usefulness of proposed treatment in arthritis.

In an embodiment of the present disclosure, the prepared ginseng oil-loaded co-processed multifunctional novel adsorbent-based transdermal patch has convincing results as compared to normal topical application of ginseng oil i.e., conventional method. It might be because of increase in contact time, presence of penetration enhancers and/or pressure of the patch. Henceforth, based on available pieces of evidence, it can be concluded that prepared patches have good potential in the management of arthritis in comparison to a conventional approach.

### Example 4. Development and Evaluation of Luliconazole Dusting Powder by s-SMEDDS Approach

In the embodiment of the present disclosure, the development of Luliconazole dusting powder by solid self-microemulsifying drug delivery system (s-SMEDDS). Luliconazole shows poor solubility, transdermal penetration and stability. A surface absorption technique was employed on luliconazole that could enhance its solubility, permeation property and stability. The formulated liquid SMEDDS was further converted into solid SMEDDS by employing surface absorption using a co-processed multifunctional novel excipient. Moreover, the solid SMEDDS have improved the permeation property and also showed better formulation stability. In vitro drug release study and permeation study showed that the prepared formulation has good permeation properties of about 48 μg/cm². In the skin irritancy test, both formulations showed no sign of edema or redness to the skin and it is concluded that the prepared formulations are suitable for topical use. The antifungal activity study showed that there is no growth of any fungal species after the study performed on the culture of A. niger and C. albicans.

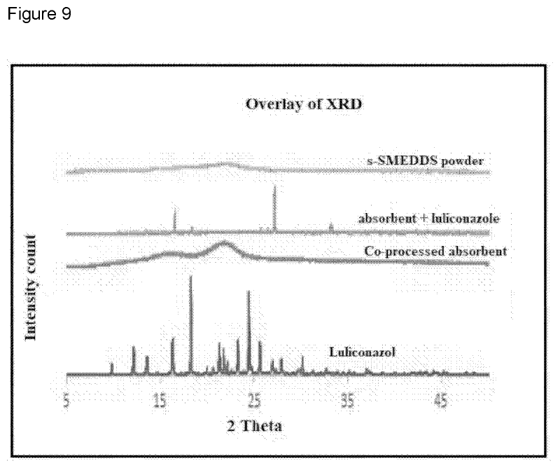

PXRD pattern of the solid SMEDDS showed absence of peaks indicating a polymorphic transition at molecular stage. The disappearance of prominent peaks relating to pure Luliconazole indicated the drastic transformation of the crystalline structure of Luliconazole into an amorphous state after preparation of solid SMEDDS (FIG. 9).

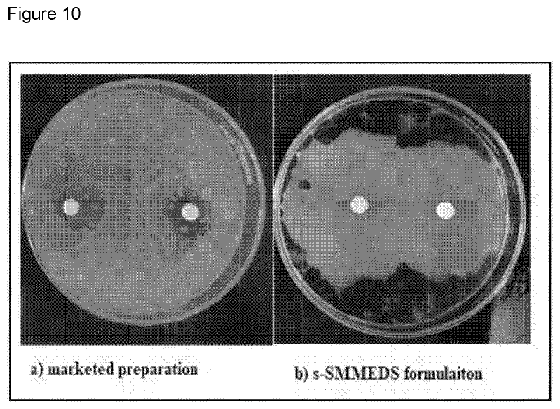

Optimized SMEDDS powder exhibited optimum antifungal activity against one of the major human fungal pathogens Candida albicans wherein the optimized powder exhibited a larger zone than the marketed one, indicating strong antifungal activity as seen in FIG. 10. The prepared formulation showed their action by inhibiting the formation of ergosterol in the cell wall of fungal species.

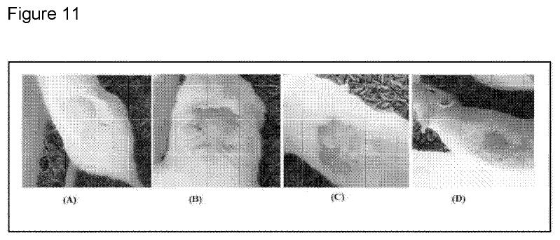

There was no evidence of any signs of erythema or edema observed post 24, 36, 48 and 72 h at site of application (FIG. 11). It showed non-irritating nature of the prepared formulation when applied to the skin. All excipients used in the formulation were therefore found to be non-irritating and safe for topical application.

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

1. A process for preparing a co-processed multifunctional excipient comprising:
(a) natural coconut husk and/or coir pith powder derived from the husk of coconut, the fruit of Cocos nucifera; and
(b) at least one polyol compound consisting of glycerol, sorbitol, polyethylene glycol (PEG), propylene glycol (PG), and combinations thereof,
wherein the weight ratio of (a) to (b) is in the range of 1:1 to 6:4,
the process comprising:
processing a mixture comprising (a) and (b), wherein the weight ratio of (a) to (b) is in the range of 1:1 to 6:4, by twin-screw hot melt extrusion under conditions in which: the ambient temperature of the twin-screw hot melt extrusion is 60-110° C; and
the screw rotation speed is 25-60 rpm, thereby producing the co-processed multifunctional excipient.

2. The process for preparing a co-processed multifunctional excipient derived from the husk of coconut, the fruit of Cocos nucifera, according to claim 1, wherein the water holding capacity of the co-processed multifunctional excipient is in the range of 6 to 10 times its own weight.

3. The process according to claim 1, wherein the coconut husk and/or coir pith powder is used as an absorbent.

4. The process according to claim 2, wherein the coconut husk and/or coir pith powder is used as an absorbent.

5. The process according to claim 1, wherein the at least one polyol and/or combinations of a plurality of polyols increase the bulk density of particles.

6. The process according to claim 2, wherein the at least one polyol and/or combinations of a plurality of polyols increase the bulk density of particles.

7. The process according to claim 1, wherein the co-processed multifunctional excipient is used as a multifunctional component such as:
an absorbent, a scavenging agent, a flow property improver, a thickening agent, and/or a viscosity enhancer.

8. The process according to claim 2, wherein the co-processed multifunctional excipient is used as a multifunctional component such as:
an absorbent, a scavenging agent, a flow property improver, a thickening agent, and/or a viscosity enhancer.
